(19) United States
(12) Patent Application Publication
(10) Pub. No.: US 2002/0132756 A1
(43) Pub. Date: Sep. 19, 2002

(54) Encapsulated oil and fat products with free fatty acids

(76) Inventor: John H. Lee, Olathe, KS (US)

Correspondence Address: John H. Lee, 1441 N. Lucy Montgomery Way Street, Olathe, KS, 66061, US

(21) Appl. No.: 09/760,971

(22) Filed: Jan. 16, 2001

(51) Int. Cl. A61K 31/715; A61K 9/48

(52) U.S. Cl. 514/2; 514/54; 424/451

(57) ABSTRACT

Encapsulated oil and fat products with protein and carbohydrate rich ingredients are provided, in which the free fatty acids in the oils and fats are required from 5 to 100%. The oils and fats can be from plant or animal source. The oils and fats with free fatty acids 5-100% may be encapsulated with protein and carbohydrate rich ingredients to form gels under a heat treatment above 65°C. The rate of the lipid against the protein may be from 30-80 % (fat) to 70-20% (protein). The encapsulated oil and fat products may be used as food and feed ingredients, which are easily handed and packaged with longer shelf life. In ruminant feed, the encapsulated products may have the by-pass function.

Inventors: Lee; John H.; 1441 N. Lucy Montgomery Way Street, Olathe, Kans. 66061 (US)

Assignee: Rigel Technology Corporation; 14440 W. 100 Street, Lenexa, Kans. 66215 (US)

Reference Cited:
U.S. Pat. Documents

| | | | |
|---|---|---|---|
| 4138505 | Feb., 1979 | Hart et al. | 426/98 |
| 4216234 | Aug., 1980 | Rawlings et al. | 426/98 |
| 4217370 | Aug., 1980 | Rawlings et al. | 426/98 |
| 4808429 | Feb., 1989 | Freeman | 426/647 |
| 5514388 | May, 1996 | Rohwer | 426/231 |

### BACKGROUND OF THE INVENTION

There are several benefits for oil and fat encapsulated products. The products can be easily handed and packaged as food and feed ingredients. The dry products can be storied for longer time. In ruminant feed, encapsulated products may pass through rumen substantially unchanged for subsequent digestion in the abomasum and lower gut of the ruminant animal, which may increase the milk production for dairy cows.

Over the years, various attempts have been made to make fat encapsulated products. A number of patents have been issued to make the encapsulated fat-protein products by heat treatment and pH change treatment.

U.S. Pat. No. 5,514,388 discloses a process to treat protein with a base to increase the pH to 9-13 at first. Then the fat is added into the alkali protein. The protein firm gel is formed when the pH is lowed to 3 to 5.

U.S. Pat. No. 4,808,429 discloses a process to encapsulate animal blood and fat by heating to a temperature in the range 40-45 degree ° C. and homogenizing into a dispersion or emulsion. Then the mixture was allowed to cool and to form a gel.

U.S. Pat. No. 4,217,370 discloses a process to treat protein with a base to increase the pH to 9-13 at first. Then the fat is added into the alkali protein. The protein and fat are mixed together before any pH change. The protein gel is formed when the pH is lowed to the isoelectric point.

U.S. Pat. No. 4,216,234 discloses a process to render a proteinaceous medium with strongly alkaline (pH 9.6-12.5) prior to the formation of the fat dispersion or emulsion, and thereafter the dispersion or emulsion should be heated to a temperature within the range 40-100° C. until a gel form.

U.S. Pat. No. 4,138,505 discloses a process to heat animal blood to a temperature in the range of from about 20 to about 60° C., to adjust the pH of the heated aqueous medium to a level ranging from 9 to about 13 to form a blood solid gel, and thereafter recovering the fat-protein gel.

The commercial products by above methods have not been found over the years because the processes are still difficult to be scaled up into bulk production. In pH change treatment, ash level may be increased in the final products because of base and acid involved in the processes. Also the formed gel by pH change is not easily controlled. There is a need for an inexpensive process, which can be easily scaled up into the production to make the oil and fat encapsulated complexes with protein or/and carbohydrate rich ingredients. It is very important to commercialize the processes into products.

It has been found by the present invention that oil or fat products with free fatty acids (5-100%) can be encapsulated easily by protein or/and carbohydrate rich ingredients through a heat treatment. The purpose of the present invention is to provide a simple heat process for the preparation of oil or fat (with free fatty acids 2 to 100%) encapsulated products. Heat process such as direct steam injection process may be a very simple, economic and practical method to form protein or/and carbohydrate gels for encapsulating oil or/and fat products.

### SUMMARY OF THE INVENTION

The present invention overcomes the problems of other patents and provides a process to form lipid-protein or lipid-protein-carbohydrate encapsulated products by the simple heat treatment.

The oils and fats can be from plant or animal source such as soy free fatty acid oil, animal fat, fish oil, conjugated linoleic acid oil and others. Oil and fat ingredients with free fatty acids at least 5% or up are required for this encapsulated process. The typical conjugated linoleic acid oil has 90-99% free fatty acids. Animal fat has 2-98% free fatty acids. Soy free fatty acid oil has 30-70% free fatty acids. These oil and fat ingredients can be from food industry. There also are other oil and fat products with wide range of free fatty acids from chemical and other industries. There are number of oil and fat products, which have free fatty acids from 5 to 100%. Conjugated linoleic acid (CLA) oil is a relative new product, which relates to human and animal health, animal meat quality and milk quality. U.S. Pat. Nos. such as 5,554,646, 5,430,006 and 5,428,072 are related to conjugated linoleic acid (CLA) products.

Protein and carbohydrate rich ingredients come from different sources such as soy, corn, wheat, plasma, animal blood, albumin fraction, yeast, whey protein concentrate, cotton seed meal, starch, polysaccharide gum and others, which may form the gel and coagulation under heat treatment. Wheat gluten, plasma, animal blood, soy and others have high protein levels, which can be used to encapsulate the oil and fat ingredients to form high protein and high fat level products. Corn meal, starch and polysaccharide gum have high carbohydrate and low protein levels, which also can be used to encapsulate the oil and fat ingredients to form high carbohydrate and fat level products. Different nutritional values can be matched by the mixture of these ingredients at certain rates.

A oil or fat ingredient is mixed with a protein or/and carbohydrate rich ingredient. For preventing product lipid oxidation, some food or feed grade chemicals may be added into the mixture. Then a heat source such as direct steam injection or indirect heat exchange may be applied into the mixture to a temperature above 65° C. to form lipid-protein or lipid-protein-carbohydrate gel.

The wet lipid-protein or lipid-protein-carbohydrate product with moisture (e.g. 40-60%) may be used in the applications without any further dry process. Also the wet lipid-protein or lipid-protein-carbohydrate product may be dried into a dry form with a common dryer. The dry product may be stored for longer time.

Two or more oil, fat, protein or carbohydrate ingredients may be mixed for preparing the oil and fat encapsulated products. Besides oil, fat, carbohydrate and protein ingredients, other ingredients or nutrients such as minerals and vitamins may also be added into the mixture for nutritional and other reasons.

The present oil and fat encapsulated process is an easy and economic process, which may make the encapsulation process easily to be commercialized.

### DETAIL DESCRIPTION OF THE PREFERRED EMBODIMENTS

The following examples set forth preferred methods in accordance with the invention. It is to be understood, however, that these examples are provided by way of illustration and nothing therein should be taken as a limitation upon the overall scope of the invention.

### EXAMPLE 1

### (Soy Meal and Conjugated Linoleic Acid)

Soy meal powder (27 grams) was mixed with water (39 grams). Then conjugated linoleic acid oil (21 grams) was mixed with the soy meal. Then mixture was mixed and heated to 80° C. with a mixer for 5 minutes. The mixture was dried in an oven at 105° C. for 3 hours. The product was yellowish color particles. The analytical data were follows: protein (31.3%), moisture (6.0%), fat (40.6%), and ash (3.8%).

### EXAMPLE 2

### (Animal Fat, Animal Blood and Corn Meal)

Corn meal powder (30 grams) was mixed with turkey whole blood (42 ml) and animal fat (20 grams). The mixture was mixed and heated to 85° C. with a mixer for 4 minutes. The mixture was dried in an oven at 105° C. for 5 hours. The analytical data were follows: protein (21.4%), moisture (2.8%), fat (32.8%), and ash (5.4%).

### EXAMPLE 3

### (Wheat Gluten and Conjugated Linoleic Acid)

Wheat gluten powder (33 grams) was mixed with 60 grams of water. Then conjugated linoleic acid oil (26 grams) was mixed with the wheat gluten. The mixture was mixed and heated to 85° C. with a mixer for 6 minutes. The mixture was dried in an oven at 105° C. for 3 hours. The resulting solid was ground into a yellowish color powder. The analytical data were follows: protein (35.7%), moisture (3.6%), fat (41.6%), and ash (2.5%).

### EXAMPLE 4

### (Cotton Seed Meal, Animal Fat and Chicken Blood)

Cotton seed meal (50 grams) was mixed with chicken whole blood (140 ml) and animal fat (60 grams). The mixture was mixed and heated to 80° C. with a mixer for 3 minutes. The mixture was dried in an oven at 105° C. for 5 hours. The analytical data of the product were follows: protein (36.4%), moisture (2.0%), fat (39.6%), and ash (5.4%).

### EXAMPLE 5

### (Plasma and Conjugated Linoleic Acid)

Bovine plasma with 18% solid (261 grams) was mixed with Rigel 1005 (0.5 grams), conjugated linoleic acid (51 grams) and Rigel 1015 (0.3 grams). The mixture was mixed and heated to 80° C. with a mixer for 6 minutes. The mixture was dried in an oven at 105° C. for 3 hours. The analytical data of the product were follows: protein (37.6%), moisture (2.3%), fat (48.7%), and ash (5.9%).

### EXAMPLE 6

### (Animal Blood and Soy Free Fatty Acid Oil)

Chicken whole blood (330 ml) was mixed with Soy Free Fatty Acid Oil (90 g), Rigel 1005 (1.2 grams), Rigel 1010 (0.5 ml) and Rigel 1003 (2.5 ml). The mixture was mixed and heated to 85° C. for 6 minutes. The mixture was dried in an oven at 105° C. for 3 hours. The analytical data were follows: protein (32.9%), moisture (3.0%), fat (55.1%), and ash (4.2%). The product was no lipid rancid smell after stored for 90 days in the summer.

What is claimed is:

1. A method of preparing oil and fat encapsulated products comprising the steps of
(a). mixing oil and fat ingredient with protein or/and carbohydrate ingredients;
(b). heating above mixture to a temperature above 65° C. for a period of time to form a firm gel product.

2. The method of claim 1, the oil and fat ingredients may have free fatty acids from 5% to 100%.

3. The method of claim 1, the protein or carbohydrate ingredients may be soy, animal blood, plasma, albumin fraction, corn, yeast, cotton seed, whey protein concentrate, starch, polysaccharide gum and others, which have the function to form the gel and coagulation under heat treatment.

4. The method of claim 1, some food or feed grade chemicals may be used for preventing lipid oxidation.

5. The method of claim 1, the process temperature is higher than 65° C. The heat source may be applied directly or indirectly such as steam injection or heat exchanger.

6. The method of claim 1, the encapsulated oil and fat product may be from one or more oil, fat, protein or carbohydrate ingredients. Also other ingredients may be added into the final product for nutritional and other reasons.

7. The method of claim 1, the encapsulated oil and fat product may be used as wet form or dry form after a dry process.

8. An encapsulated oil or fat product is prepared by the method of claim 1.
